(19) United States
(12) Patent Application Publication
(10) Pub. No.: US 2005/0247515 A1
(43) Pub. Date: Nov. 10, 2005

(54) Ear-plug device, method for manufacturing the same and use of such an ear-plug device

(75) Inventor: Christian Berg, Uerikon (CH)

Correspondence Address: NIXON PEABODY, LLP, 401 9TH STREET, NW, SUITE 900, WASHINGTON, DC, 20004-2128, US

(73) Assignee: Phonak AG, Staefa (CH)

(21) Appl. No.: 10/841,830

(22) Filed: May 10, 2004

(51) Int. Cl. A61B 7/02

(52) U.S. Cl. 181/135

(57) ABSTRACT

The invention relates to an ear-plug device adapted for being worn at least in part in an ear canal of a person, comprising a hollow rigid hard shell made of a first material and a resilient soft suspension portion fixed to the hard shell and made of a second material less rigid than the first material, said hard shell having an outer surface individually shaped according to the specific shape of said person's ear canal, said soft suspension portion being adapted to receive an electro-acoustic output transducer in a detachable manner within an outer opening of said soft suspension portion, with said soft suspension portion being at least partly surrounded by said hard shell.

### BACKGROUND OF THE INVENTION

1. Field of the Invention

The present invention relates to an ear-plug device for being worn at least in part in the ear canal of a person, a method for manufacturing such an ear-plug device and a use of such an ear-plug device.

2. Description of Related Art

WO 02/071794 A1 discloses earpieces to be worn in the ear canal of a person which may act as housings for hearing aids, wireless or connected communication devices such as headsets, mobile phones, personal agents, loudspeakers, tinnitus masking devices and others. The earpieces are shaped by computer-assisted modeling according to the individual shape of the person's ear canal which is obtained by three dimensional scanning of the person's ear canal. The earpiece comprises a rigid hard shell which houses the electrical components which are mounted on a plate which is fixedly connected to the shell. Such earpieces have the drawback that for each audio device a separate hard shell is needed.

DE 100 46 202 A1 discloses an ear-plug device comprising a soft shell made of silicon having a hardness of 30 to 90 shore. The soft shell is shaped according to the individual shape of the person's ear canal from an impression the person's ear canal and includes an acoustic canal which opens into the person's ear canal. At the other end of the acoustic canal a speaker is received by the soft shell. The inserted speaker is connected by wires or in a wireless manner to a communication device such as a mobile phone. The soft shell is worn in the person's ear canal.

Ear-plug devices comprising an elastic shell with an integrated speaker to be worn in the person's ear canal are also described, for example, in GB 2 373 951 A, DE 101 40 369 A1 and DE 100 38 825 A1. However, these ear-plug devices are not individually shaped according to the shape of the person's ear canal.

In general, the use of soft materials for an ear-plug device involves problems regarding durability, elasticity change over time, coloring, lack of device protection etc.

The use of hard materials for ear-plug devices—unless customized to the individual person's ear shape—involves problems with the individual fit and retention of the device, since the distribution of ear shapes is extremely flat and few people have “the average ear”, which results in uncomfortable wearing properties already after short usage.

WO 03/069951 A1 discloses an ear clasp headset including a speaker capsule, wherein a clasp is added to a standardized earphone in order to hook onto the flip of the ear.

US 2003/0133583 A1 discloses an ear-plug device for being worn in the ear canal of a person which comprises a hollow shell which accommodates the electrical components of an in-the-ear hearing aid. The shell is made of a rubber-like elastic material and can be slipped over the electronic components for exchanging the shell if necessary, for example, due to changes of the shape of the person's ear canal. The shell is shaped according to the determined shape of the person's ear canal and is manufactured by an incremental layer-by-layer build-up process, such as selective laser sintering of a powder material. The shell may comprise a distal section which is made of a more rigid material than the proximal rubber-like elastic section accommodating the electronic components. Such structure may be achieved by changing the material during the incremental layer-by-layer build-up process.

It is an object of the invention to provide for an ear-plug device for holding an electric-acoustic output transducer close to or within the a person's ear canal, wherein a comfortable and reliable fit within the ear canal should be ensured and wherein the same ear-plug device should be usable for different electro-acoustic output transducers in a comfortable and reliable manner.

It is a further object to provide for a method for manufacturing such an ear-plug device.

It is still a further object of the invention to provide for a use of such an ear-plug device.

### SUMMARY OF THE INVENTION

The above objects are achieved by an ear-plug device as defined in claims 1 and 28, respectively, a corresponding manufacturing method as defined in claims 34 and 40, respectively, and a use as defined in claims 41 and 43, respectively.

The solution according to the invention in general is beneficial in that on one hand, by employing a hollow rigid hard shell having an outer surface individually shaped according to the specific shape of person's ear canal, a high wearing comfort and reliable retention within the ear canal is achieved without encountering the drawbacks of the use of soft materials such as low durability, high elasticity change over time, lack of device protection etc., while on the other hand, by employing a resilient soft suspension portion fixed to the hard shell, different electro-acoustic output transducers may be fixed to the ear-plug device in a detachable (i.e. exchangeable) manner.

The solution of claims 1 and 34 is beneficial in that, since the hard shell at least partially surrounds the soft suspension portion, the respective electro-acoustic output transducer may be fixed to and released from the ear-plug device in a particularly simple, comfortable and reliable manner.

The solution of claims 28 and 40 is beneficial in that, since the soft suspension portion and the hard shell are connected in a detachable manner, the soft suspension portion can be easily exchanged, whereby flexibility of the ear-plug device is enhanced.

Preferably, the ear-plug device is designed such that the electro-acoustic output transducer may penetrate into the outer opening of the hard shell for achieving a particularly reliable retention of the transducer at the ear-plug device. Preferably, the ear-plug device is adapted for snap retention of the transducer within the soft suspension portion. Thereby a particularly simple fixing/release of the transducer at/from the ear-plug device is achieved.

The soft suspension portion may be detachable from the hard shell, whereby the hard shell preferably is adapted for snap retention of the soft suspension portion within outer opening of the hard shell. To this end, the outer opening of the hard shell may have a smaller inner diameter than the adjacent part of the hard shell.

Preferably, the output transducer is an ear-phone of a mobile phone, a MP3 player, a CD-player or a mini-disc player or a similar audio or communication device.

Preferably, the hard shell is built-up by an additive process (“rapid prototyping”), such as by a layer-by-layer laser sintering process of a powder material such as polyamide.

These and further objects, features and advantages of the present invention will become apparent from the following description when taken in connection with the accompanying drawings which, for purposes of illustration only, show several embodiments in accordance with the present invention.

### BRIEF DESCRIPTION OF THE DRAWINGS

FIG. 1 shows a schematic cross-sectional view of an example of an ear-plug device according to the invention when worn in the ear canal of a person, with an earphone being shown in engaged condition with the ear-plug device;

FIG. 2 shows a schematic view seen along the arrow II of FIG. 1.

FIG. 3 shows a schematic perspective view of an example of the use of an ear-plug device according to the invention with snap retention of the soft suspension portion within the hard shell;

FIG. 4 shows a view like FIG. 3, wherein, however, the soft suspension portion is fixed to the hard shell by a bayonet connection;

FIG. 5 shows a view like FIG. 3, wherein, however, the soft suspension portion comprises a hook for improved snap retention of an earphone;

FIG. 6 shows a view like FIG. 3, wherein, however, the soft suspension portion is fixed at the hard shell by sliding engagement within a guide track provided at the hard shell;

FIG. 7 shows a view like FIG. 3, wherein, however, an earphone is to be inserted into the hard shell through a lateral opening of the hard shell, and wherein the ear-plug device is seen from below; and

FIG. 8 shows a view like FIG. 3, wherein the example of FIG. 7 is shown in a mounted state.

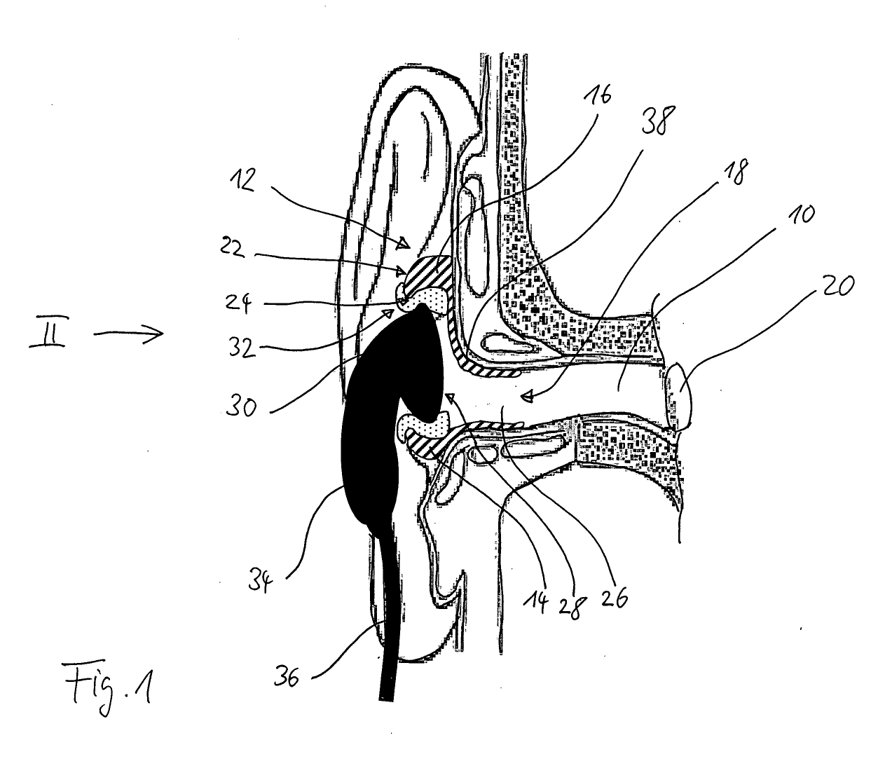

FIG. 1 shows a cross-sectional view of a person's ear in the region of the ear canal 10, with an ear-plug device 12 being inserted in the outer portion of the ear canal 10. Preferably, the ear-plug device 12 is designed such as to minimize the necessary ear canal penetration. The outer end of the ear-plug device 12 projects outwardly from the ear canal 10.

The ear plug device 12 comprises a hollow rigid hard shell 14 made of a first material and a resilient soft suspension portion 16 fixed to the hard shell 14 and made of a second material less rigid than the first material. The hard shell 14 has an inner end with an inner opening 18 facing the ear drum 20 and an outer end 22 with an outer opening 24. The hard shell 14 is tube-like and comprises an acoustic canal 26 extending between the inner opening 18 and the outer opening 24.

The soft suspension portion 16 has ring-like shape and comprises an acoustic canal 28 which is acoustically connected to the acoustic canal 26 of the hard shell 14. The term “ring-like shape” is intended to cover any type of torus-shaped elements and is not restricted to circular elements. The soft suspension portion 16 penetrates through the outer opening 24 of the hard shell 14 and is located mainly within the interior, i.e. the acoustic canal 26, of the hard shell 14. According to FIG. 1 only a small portion of the soft suspension portion 16 extends outwardly beyond the hard shell 14.

The soft suspension portion 16 is preferably made of silicone rubber and preferably has an elasticity of from shore A 75 to shore A 45, while the hard shell 14 preferably is made of nylon and has an elasticity of from shore D 85 to shore D 70.

The soft suspension portion 16 is adapted to receive an electro-acoustic output transducer in a detachable, i.e. exchangeable or removable, manner due to its elasticity and due to the fact that the soft suspension portion 16 extends through the outer opening 24 of hard shell 14.

The soft suspension portion 16 preferably is fixed to hard shell 14 in a detachable manner, preferably by snap retention of the soft suspension portion 16 within the outer opening 24 of the hard shell 14. To this end, the example of FIG. 1 the outer opening 24 of the hard shell 14 has a smaller inner diameter than the adjacent part of the hard shell 14.

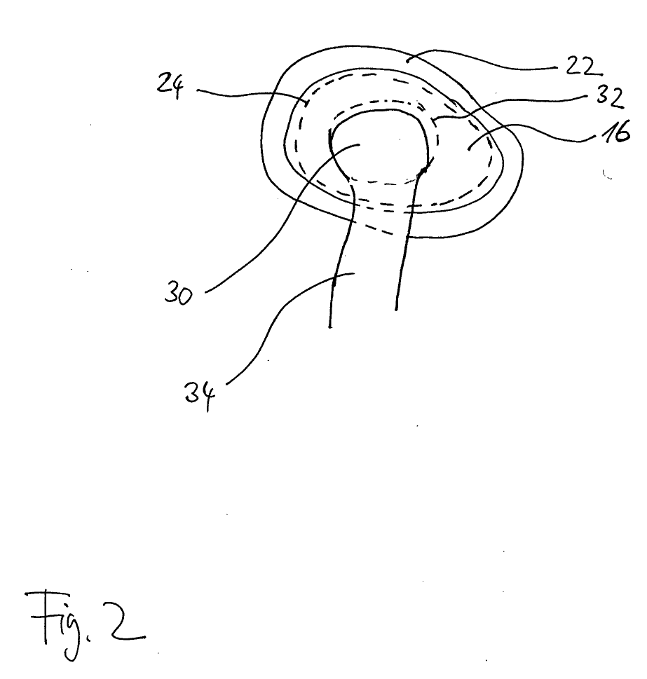

FIG. 3 shows schematically how the ring-like soft suspension portion 16 is moved into the outer opening 24 of the hard shell 14 for achieving a snap retention of the soft suspension portion 16 within the hard shell 14.

In FIG. 4 an alternative embodiment is shown schematically, wherein the soft suspension portion 16 is provided at its periphery with bayonet engagement elements 40 for achieving a bayonet connection with mating bayonet counterpart elements 42 provided at the hard shell 14 at or close to the outer opening 24. The bayonet connection is opened or closed by rotating the soft suspension portion 40 and the hard shell 14 relative to each other, as indicated in FIG. 4.

By using a bayonet connection as shown in FIG. 4 rather than a snap connection as shown in FIG. 3 the material of the suspension portion 16 may be harder (i.e. less elastic) than in the case of a snap connection.

In FIG. 6 an example is shown, wherein the soft suspension portion 16 is fixed to the hard shell 14 by a slide engagement. The soft suspension portion 16 is provided with slide engagement elements 44, while the hard shell 14 is provided with guide track elements 46 at or close to the outer opening 24 of the hard shell 14. The guide track elements 46 are adapted to receive the slide engagement elements 44 in such a manner that the soft suspension portion 16 is fixed with regard to the longitudinal axis of the hard shell 14 while it is movable in a direction perpendicular to the longitudinal axis of the hard shell 14, as shown in FIG. 6. Also in this embodiment, the material of the soft suspension portion 16 may be harder, i.e. less elastic, than in the embodiment of FIG. 3.

By providing for such a detachable fixation, the soft suspension portion 16 may be replaced either by a new soft suspension portion 16 of the same type for avoiding excessive degradation of the soft suspension portion 16 or by a soft suspension portion 16 of a different type being adapted for receiving one out of a plurality of differently shaped electro-acoustic output transducers 30. In this respect, the inner surface of the hard shell 14 may provide for a “standard shape” to which at least part of the outer surface of the different types of soft suspension portions 16 adapted.

The term “electro-acoustic output transducer” is intended to cover all transducers which convert an electrical signal into sound. Preferably, the output transducer is an earphone 30 of, for example, a mobile phone, a MP3 player, a CD player or a mini-disc player or a similar communication or audio device.

Preferably the ear-plug device 12 is adapted for a snap engagement or retention of the earphone 30. To this end, the outer opening 24 has a smaller inner diameter than the adjacent part of the hard shell 14 and the earphone 30 is able to penetrate into or through the outer opening 24 of the hard shell 14, as shown in FIG. 1.

Thereby the earphone 30 may be engaged with the ear-plug device 12 by the user simply pressing the earphone 30 into the outer opening 32 of the soft suspension portion 16 until snap retention of the ear phone 30 is achieved. In addition, thereby reliable retention of the earphone 30 within the ear-plug device 12 is achieved. The earphone 30 may be released from the ear-plug device 12 by the user drawing an elongated portion 34 of the ear phone outwardly until the snap retention within the outer opening 32 of the soft suspension portion 16 is released.

Also in the embodiments of FIGS. 3, 4 and 6 the earphone 30 is fixed to the soft suspension portion 16 by snap engagement by pressing the earphone 30 into the outer opening of the suspension portion 16.

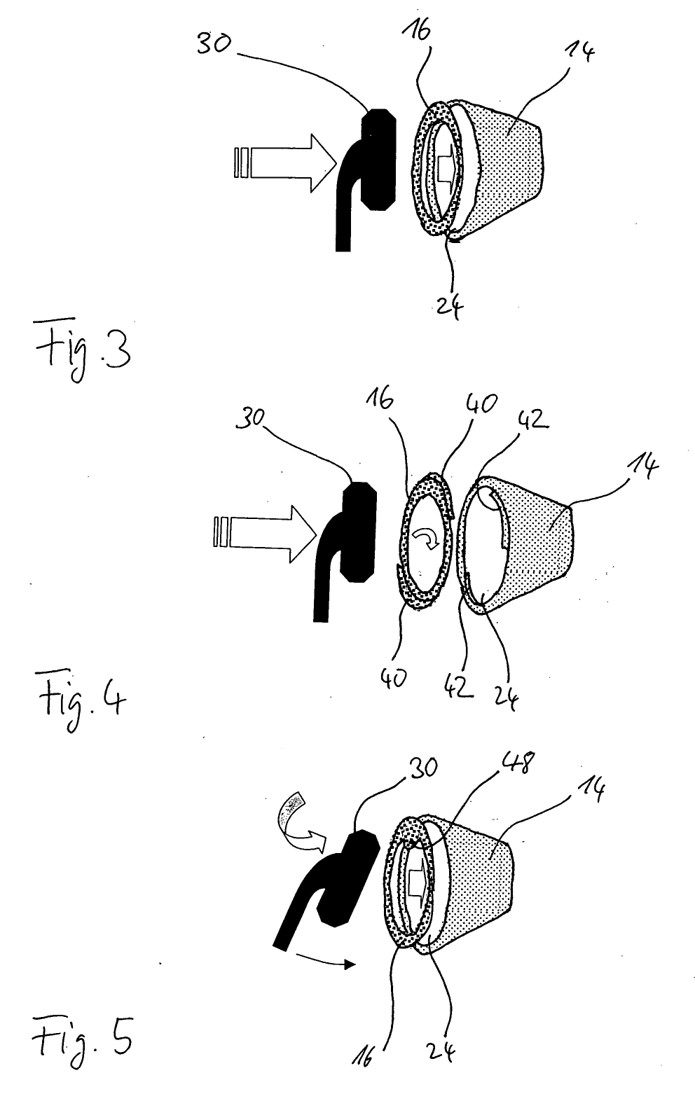

FIG. 5 shows schematically a modified embodiment, wherein the soft suspension portion 16 is provided with a hook element 48 which projects into the outer opening 32 of the soft suspension portion 16 in the uppermost part of the outer opening 32. The soft suspension portion 16 is fixed to the hard shell 14 by snap engagement, as in FIG. 3. The hook element 48 serves to improve the snap retention of the earphone 30 within the outer opening 32. To this end, the earphone 30 is inserted into the outer opening 32 starting from a tilted position (tilted relative to vertical) as shown in FIG. 5, with the upper end of the earphone 30 being positioned behind (i.e. inwardly) of the hook element 48. Thereafter the lower portion of the earphone is pressed into the outer opening 32, whereby the earphone 30 rotates around an axis perpendicular to the paper plane of FIG. 5 (see arrow at the bottom of FIG. 5). The hook element 48 is provided at the upper portion of the outer opening 32, since, when the earphone 30 is in its engaged position with the soft suspension portion 16, gravity acts in such a manner on the earphone 30 that the upper end experiences an outwardly directed force while the lower end experiences an inwardly acting force (inwardly/outwardly is meant relative to the ear canal). Hence, the hook element 48 prevents the earphone from tilting and falling out of the soft suspension portion 16 according to the gravity-induced forces.

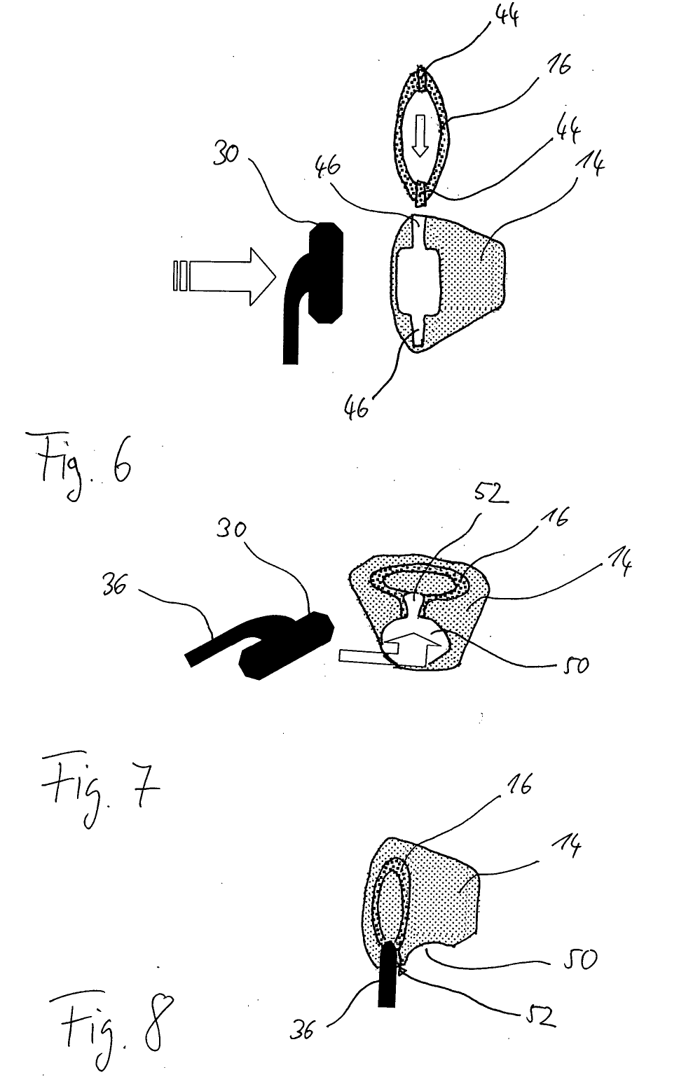

In FIGS. 7 and 8 an embodiment is shown, wherein the earphone 30—in contrast to the embodiments described so far—is not pressed from outside into the outer opening 32 of the soft suspension portion 16 but rather is pressed from the inside of the hard shell 14 outwardly in order to engage with the soft suspension portion 16, e.g. by snap retention. To this end, the hard shell 14 is provided with an opening 50 at its lower portion. The opening 50 is designed such that the earphone 30 can be passed through the opening 50 into the interior of the hard shell 14. An outwardly directed elongated portion 52 of the opening 50 enables the elongated portion 34 of the earphone 30 to move outwardly when the earphone 30 is pressed into the soft suspension portion 16. In this embodiment, the soft suspension portion 16 is not a closed ring but rather includes a cut-out portion at the elongated opening 52 through which the elongated portion 34 of the earphone of the earphone 30 can travel when the earphone 30 is pressed into the soft suspension portion 16. FIG. 8 shows the mounted state of the earphone 30.

In the embodiment of FIGS. 7 and 8, the soft suspension portion 16 may be located completely within the hard shell 14 and may be fixedly connected to the hard shell 14 or it may be detachably connected to the hard shell 14.

The ear-plug device 12 may serve to subsequently receive different earphones 30 having close to the same outer dimensions and shapes or to subsequently receive different earphones 30 having different outer dimensions and shapes. Thereby the same ear-plug device 12 may be used for a plurality of earphones 30.

With the soft suspension portion 16 being fixable within the hard shell 14, there are two alternative manners of fixing a given earphone 30 to the ear-plug device 12: either the soft suspension portion 16 is first fixed within the hard shell 14 and then the earphone 30 is fixed within the soft suspension portion 16 or the earphone 30 is first fixed within the soft suspension portion 16 and then the soft suspension portion 16 together with the earphone 30 is fixed within the hard shell 14.

The earphone 30 may be connected to the respective audio or communication device by wires 36 or by a wireless link.

The ear-plug device 12 usually is only worn at times when use of the ear phone 30 is desired. At other times, the ear-plug device 12 may be removed from the ear canal 10.

Generally, the outer, i.e. circumferential, surface 38 of the hard shell 14 is individually shaped according to the specific shape of the ear canal 10. To this end, prior to manufacturing the individual shape of the ear canal 10 has to be determined, for example, by direct three-dimensional laser scanning of the ear canal 10 and the concha or by producing an impression the ear canal 10 and the concha. In the first case, digital data of the shape of the ear canal 10 and the concha is directly determined while in the latter case such data is obtained from the impression, e.g. by three-dimensional scanning of the impression.

The digital data obtained thereby then may be used to create the hard shell 14 by an additive build-up process. Such additive or incremental building processes are also known as “rapid prototyping”. An overview regarding such processes can be found, for example, in US 2003/0133583 A1.

A preferred incremental additive build-up process is a layer-by-layer laser sintering process of a powder material. Such processes are also designated as “selective laser sintering” (SLS). A thin layer of hot melting powder is applied on a powder bed, for instance, by means of a roller. A laser beam, controlled by the three-dimensional data of the ear canal 10, solidifies the powder layer which corresponds to a slice or sectional layer of the hard shell 14. A solid sectional layer of the hard shell 14 is thus produced in the otherwise loose powder. That layer is then lowered out of the powder deposition plane and a new powder layer is superposed, laser-solidified to constitute another sectional layer, etc. Thus the basic principle in the incremental build-up process consists in the deposition of a thin layer of material on a surface, with the desired sectional shape then being stabilized, i.e. hardened.

Once a layer has hardened, a new layer is deposited on it, hardened and bonded to the finished layer underneath. In that fashion, layer by layer, the hard shell 14 is composed by the successive, additive deposition of multiple layers.

In commercial production, the preferred method is to deposit and solidify layers of several hard shells 14 in parallel by using a single laser beam. A single laser solidifies the sectional layers of several hard shells 14 before all hardened sectional layers are jointly dipped. Thereupon, after a new powder layer has been deposited on all hardened and dipped sectional layers, the next multiple sectional layers are formed. Although fabricated in parallel, the individual hard shells 14 are produced as separate units under appropriate digital control. Rather than using a single laser beam, also more than one laser beam may be operated and controlled in parallel.

An alternative process to selective laser sintering is laser or stereo lithography, wherein a first sectional layer of the hard shell 14 is solidified on the surface of a liquid photopolymer by means of a UV laser. The hardened layer is dipped and again covered with the liquid polymer. By means of the UV laser the second sectional layer of the hard shell 14 is solidified on the first hardened layer and so on.

A further alternative process is the thermojet process wherein the contouring for a given sectional layer of the hard shell 14 follows a principle similar to that of an ink jet printer, in that liquid is applied based on the three-dimensional data of the ear canal 10. The sectional image deposited is then allowed to solidify. Again following the principle of an incremental build up, layer upon layer is deposited in building the hard shell 14.

A preferred process is available under the trademark NemoTech from applicant, wherein an impression of the ear canal is taken and then undergoes three-dimensional laser scanning with high resolution. The thus obtained three-dimensional data undergo shell modeling for determining the best possible fit. Then the hard shell 14 is produced by selective laser sintering from polyamide powder. Subsequently the outer surface of the hard shell 14 is smoothed and provided with a skin-line texture to ensure a firm fit and good retention in the ear canal.

While various embodiments in accordance with the present invention have been shown and described, it is understood that the invention is not limited thereto, and is susceptible to numerous changes and modifications as known to those skilled in the art. Therefore, this invention is not limited to the details shown and described herein, and includes all such changes and modifications as encompassed by the scope of the appended claims.

1. An ear-plug device adapted for being worn at least in part in an ear canal of a person, comprising a hollow rigid hard shell made of a first material and a resilient soft suspension portion fixed to the hard shell and made of a second material less rigid than the first material, said hard shell having an outer surface individually shaped according to the specific shape of said person's ear canal, said soft suspension portion being adapted to receive an electro-acoustic output transducer in a detachable manner within an outer opening of said soft suspension portion, with said soft suspension portion being at least partly surrounded by said hard shell.

2. The device of claim 1, wherein said soft suspension portion is adapted to extend through an outer opening of said hard shell.

3. The device of claim 2, wherein said ear-plug device is designed such that said electro-acoustic output transducer may penetrate into said outer opening of said hard shell.

4. The device of claim 3, wherein said soft suspension portion has a ring-like shape.

5. The device of claim 2, wherein said hard shell comprises an inner end with an inner opening for facing an eardrum, an outer end with said outer opening and an acoustic canal extending between said inner opening and said outer opening.

6. The device of claim 5, wherein said soft suspension portion comprises an acoustic canal acoustically connected to said acoustic canal of the hard shell, with said output transducer being received at an outer end of said acoustic canal of said soft suspension portion, said outer opening of said soft suspension portion forming said outer end of said acoustic canal of said soft suspension portion.

7. The device of one claim 1, wherein said soft suspension portion is located mainly within an interior of said hard shell.

8. The device of claim 1, wherein said soft suspension portion is adapted to receive alternatively one out of a plurality of electro-acoustic output transducers of different shapes.

9. The device of claim 1, wherein said ear-plug device is adapted for snap retention of said output transducer within said outer opening of said soft suspension portion.

10. The device of claim 9, wherein said soft suspension portion is provided with a hook element.

11. The device of claim 10, wherein said hook element is provided at an upper end of said outer opening of said soft suspension portion for engagement with an upper end of said output transducer.

12. The device of claim 1, wherein said soft suspension portion is detachable from said hard shell.

13. The device of claim 12, wherein said hard shell is adapted for snap retention of said soft suspension portion within said outer opening of said hard shell.

14. The device of claim 13, wherein said outer opening of said hard shell has a smaller inner diameter than an adjacent part of said hard shell.

15. The device of claim 12, wherein said soft suspension portion is fixed to said hard shell by a bayonet connection.

16. The device of claim 12, wherein said hard shell is adapted to receive alternatively one out of a plurality of different soft suspension portions each adapted for receiving one out of a plurality of differently shaped electro-acoustic output transducers.

17. The device of claim 1, wherein said output transducer is an earphone of an audio device or a communication device, such as a mobile phone, a MP3 player, a CD player or a mini-disk player.

18. The device of claim 1, wherein said soft suspension portion is made of silicone rubber.

19. The device of claim 1, wherein said soft suspension portion has an elasticity of from shore A 75 to shore A 45.

20. The device of claim 1, wherein said hard shell has an elasticity of from shore D 85 to shore D 70.

21. The device of claim 1, wherein said hard shell is made of polyamide.

22. The device of claim 1, wherein said hard shell is a result from an additive build-up process.

23. The device of claim 22, wherein said hard shell is the result of a layer-by-layer laser sintering of a powder material.

24. The device of claim 1, wherein said outer surface of said hard shell is provided with a skin-like texture.

25. The device of claim 1, wherein said hard shell is provided with a lateral opening for inserting said output transducer into an interior of said hard shell for engagement with said outer opening of said soft suspension portion.

26. The device of claim 25, wherein said lateral opening is provided at a lower side of said hard shell.

27. The device of claim 26, wherein said lateral opening includes an elongated portion for enabling movement of said output transducer along a longitudinal axis of said hard shell for engagement with said outer opening of said soft suspension portion.

28. An ear-plug device adapted for being worn at least in part in an ear canal of a person, comprising a hollow rigid hard shell made of a first material and a resilient soft suspension portion fixed to the hard shell and made of a second material less rigid than the first material, said hard shell having an outer surface individually shaped according to the specific shape of said person's ear canal, said soft suspension portion being adapted to receive an electro-acoustic output transducer in a detachable manner within an outer opening of said soft suspension portion, wherein said soft suspension portion is detachable from said hard shell.

29. The device of claim 28, wherein said hard shell is adapted for snap retention of said soft suspension portion within said outer opening of said hard shell.

30. The device of claim 28, wherein said soft suspension portion is adapted to be fixed to said hard shell by a bayonet connection.

31. The device of claim 28, wherein said soft suspension portion is adapted to be fixed to said hard shell by a slide connection.

32. The device of claim 31, wherein said soft suspension portion is provided with slide engagement elements in sliding engagement with guide track elements provided at said hard shell.

33. The device of claim 33, wherein said soft suspension portion is adapted for being slideable in a direction perpendicular to a longitudinal axis of said hard shell.

34. A method for manufacturing an ear-plug device adapted for being at least partially worn in an ear canal of a person, comprising: determining an inner shape of said person's ear canal and an outer ear anatomy, forming a hollow rigid hard shell which is individually shaped according to said determined inner shape of said person's ear, and fixing a resilient soft suspension portion to said hard shell, said soft suspension portion being at least partially surrounded said hard shell and being adapted to receive an electro-acoustic output transducer in an outer opening of said soft suspension portion in a detachable manner.

35. The method of claim 34, wherein said soft suspension portion is fixed to said hard shell in a detachable manner.

36. The method of claim 32, wherein said soft suspension portion is fixed within said outer opening of said hard shell by snap retention.

37. The method of claim 34, wherein said hard shell is built-up by an additive process.

38. The method of claim 37, wherein said hard shell is formed by layer-by-layer laser sintering of a powder material.

39. The method of claim 34, wherein said outer surface of said hard shell after built-up is provided with a skin-like texture.

40. A method for manufacturing an ear-plug device for being at least partially worn in an ear canal (10) of a person, comprising: determining an inner shape of said person's ear canal and an outer ear anatomy, forming a hollow rigid hard shell which is individually shaped according to said determined inner shape of said person's ear, and fixing a resilient soft suspension portion to said hard shell in a detachable manner, said soft suspension portion being adapted to receive an electro-acoustic output transducer in an outer opening of said soft suspension portion in a detachable manner.

41. A use of an ear-plug device according to claim 1, comprising: fixing said soft suspension portion at least partly within said hard shell, engaging an electro-acoustic output transducer in a detachable manner with said outer opening of said soft suspension portion, inserting the ear-plug device into said person's ear, removing said ear-plug device from said person's ear, and detaching said output transducer from said soft suspension portion.

42. The use of claim 41, wherein said soft suspension portion is fixed in a detachable manner within said outer opening of said hard shell, and wherein said soft suspension portion is detached from said hard shell prior to detaching said output transducer from said soft suspension portion.

43. A use of an ear-plug device according to claim 1, comprising: engaging an electro-acoustic output transducer in a detachable manner with said outer opening of said soft suspension portion, fixing said soft suspension portion in a detachable manner at least partly within said hard shell, inserting the ear-plug device into said person's ear, removing the device from the person's ear, detaching said soft suspension portion from said hard shell, and detaching the output transducer from the soft suspension portion.

44. The use of claim 42, further comprising: replacing said soft suspension portion by a soft suspension portion or the same type or of a different shape.
